(19) United States
(12) Patent Application Publication
(10) Pub. No.: US 2010/0331286 A1
(43) Pub. Date: Dec. 30, 2010

(54) Combination therapy for treatment of bone and mineral disorders for patients with impaired renal function

(76) Inventor: Ray Chow, Newbury Park, CA (US)

Correspondence Address: Craig A Crandall, APC, 3034 Deer Valley Avenue, Newbury Park, CA, 91320, US

(21) Appl. No.: 12/806,967

(22) Filed: Aug. 25, 2010

Related U.S. Application Data
(63) Continuation-in-part of application No. PCT/US2009/001151, filed on Feb. 24, 2009.
(60) Provisional application No. 61/066,958, filed on Feb. 25, 2008.
(60) Provisional application No. 61/339,970, filed on Mar. 10, 2010.

(51) Int. Cl. A61K 31/593; A61K 31/59; A61P 29/00; A61P 19/08; A61P 3/00; B65D 83/00

(52) U.S. Cl. 514/168; 206/531

(57) ABSTRACT

The field of the present invention relates to novel and improved therapies for treatment of complications associated with impaired renal function. More specifically, the present invention relates to evaluating the effects of a novel renal multivitamin on Vitamin D levels, EPO dose, inflammatory (e.g., C-reactive protein) and other biomarkers in an end stage renal disease (ESRD) population on hemodialysis (HD).

### RELATED PATENT APPLICATIONS

This application is a continuation-in-part application of PCT/US2009/001151 (WO 2009/108297), filed on Feb. 24, 2009, which claims priority to and benefit of U.S. Provisional Application Ser. No. 61/066,958, filed Feb. 25, 2008, each of which are incorporated in its entirety by reference herein. This application also relates to and claims priority to and benefit of U.S. Provisional Application No. 61/339,970, filed Mar. 10, 2010, also incorporated in its entirety by reference herein.

### TECHNICAL FIELD

The field of the present invention relates to novel compositions and improved therapies for treatment of complications associated with impaired renal function. More specifically, the present invention relates to evaluating the effects of nutritional supplements, e.g., novel renal multivitamins, on Vitamin D levels, EPO dose, inflammatory (e.g., C-reactive protein) and other biomarkers in an end stage renal disease (ESRD) population on hemodialysis (HD).

### BACKGROUND OF THE INVENTION

Renal failure refers to temporary or permanent damage to the kidneys that result in loss of normal kidney function. There are two different types of renal failure—acute and chronic. The causes, symptoms, treatments, and outcomes of acute and chronic renal failure are different. Acute renal failure (ARF) has an abrupt onset and is potentially reversible. Chronic renal failure (CRF) is a progressive loss of renal function over a period of months or years through five stages and can lead to chronic kidney disease (CKD). Patients with CKD represent an important segment of the population (7-10%) and, mostly because of the high risk of cardiovascular complications associated with renal insufficiency, detection and treatment of CKD is now a public health priority.

In Stage 5 CKD, also known as end-stage renal disease (ESRD), the kidneys permanently fail to work. As a result of impaired renal function, the body is no longer able to maintain its internal equilibrium of water and minerals (sodium, potassium, chloride, calcium, phosphorus, magnesium, sulfate); maintain suffcient red blood cell or hemoglobin levels (anemia); or adequately remove from the blood the daily metabolic load of fixed hydrogen ions. ESRD is a severe illness and ESRD patients require renal replacement therapy, in the form of a kidney transplant or hemodialysis (HD). In the HD setting, replacement of erythropoietin and vitamin D3, two hormones processed by the kidney, is usually necessary, as is calcium replacement.

The core of anemia in CKD is impaired production of erythropoietin as well as inadequate response of erythroid marrow resulting in functional or relative iron deficiency. It is also associated with increased red blood cell fragility and hepcidin mediated iron-restricted erythropoiesis and erythropoiesis-stimulating agents (ESAs) hyporesponsiveness. In long-term HD patients, low iron stores, hyperparathyroidism, and high-turnover bone disease exacerbated by chronic inflammation and malnutrition are associated with significant ESA hyporesponsiveness. Identification of predictors of hyporesponsiveness to ESAs in HD patients is needed to help improve anemia management and reduce hemoglobin level variability.

Lack of vitamin D is a complication associated with ESRD and which may lead to increased phosphate accumulation in the body. Oral vitamin D is activated to 1, 25 OH vitamin D predominantly in the kidney. Consequently, as renal function declines, this activation process becomes increasingly less efficient. In the absence of activated vitamin D, the body may develop hyperparathyroidism (HPT), which in turn may increase serum phosphate content as a result of bone resorption. In addition to the kidney, there are other areas in the periphery of the body which may convert inactive vitamin D to active 1, 25 OH vitamin D and researchers have speculated that low dose oral supplementation of vitamin D may also be effective. As such, patients are often given 1, 25 OH vitamin D, either orally or intravenously.

The effectiveness of various vitamin D prohormones has been reviewed and reported; see, e.g., Fournier et al., Neph Dial Trans 21(10):2987-2989, 2006 (cinacalcet approach to HPT therapy); Saab et al., Nephron Clin Pract. 105(3):c132-138, 2007; Shah et al., Perit Dial Int. 25(4):362-366, 2005 (ergocalciferol therapy in hemodialysis patients); and U.S. Patent Application No. 20070122477 (Bishop et al.)(cholecalciferol and ergocalciferol to treat 25-hydroxyvitamin D insufficiency or deficiency). The K/DOQI Clinical Practice Guidelines currently recommend various ergocalciferol and/or calcitrol therapies, depending on the age of the patient and CKD stage. Unfortunately, one side effect of all vitamin D supplements is increased enteric absorption of phosphate, thus leading to hyperphosphatemia. Additional side effects include one or more of nausea, vomiting, polyuria, hypercalciuria, and hypercalcemia. Importantly, it's clear from a review of these studies and guidelines that the optimal dose or duration of vitamin D necessary to replete and maintain stores in dialysis patients is yet to be conclusively defined and/or determined. Moreover, it has yet to be conclusively determined whether such long term treatment is safe or beneficial for these patients. The need exists for further studies and therapy options.

ESRD is also associated with calcium and phosphate metabolism abnormalities that can result in severe bone disease and ectopic calcification of cardiovascular tissues; see, e.g., Slatopolsky et al., Kidney Int 4:141-145, 1973; Delmez et al., Am J Kidney Dis 19:303-317, 1992. Ectopic calcification (deposition of calcium crystals in tissues other than teeth or bone) is calcification of internal organs, including the lung, heart, stomach and kidneys and is a frequent finding in patients undergoing long-term hemodialysis. In rare instances, hemodialysis patients develop painful calcified skin lesions that progress to non-healing ulcers or gangrene and may require amputation of the affected limb.

Hyperphosphatemia (an electrolyte disturbance in which there is an abnormally elevated level of phosphate in the blood) is an important risk factor for the development of ectopic calcification and cardiovascular changes in patients undergoing hemodialysis. Often, calcium levels are lowered (hypocalcemia) due to precipitation of phosphate with the calcium in tissues. Phosphorus-restricted diets are thus essential for the prevention of these deleterious complications in ESRD patients, and patients on dialysis are given oral phosphate binders to absorb phosphate prior to its absorption from the GI tract.

The most common phosphate binders contain aluminum, synthetic resin, or calcium. And while considered essential for avoiding hyperphosphatemia, these aluminum- or calcium-based phosphate binders are associated with certain adverse effects. For example, prolonged use of aluminum gels, e.g., Amphojel®, leads to aluminum accumulation in the tissues and causes neurologic, skeletal, and hematologic toxicities, accompanied by such symptoms as encephalopathy, osteomalacia, and myopathy; see, e.g., Alfrey, et al., N Engl J Med 294:184-188, 1976; Ott et al., N Engl J Med 307:709-713, 1982. Ingestion of calcium carbonate, an effective phosphate binder, leads to hypercalcemia and increases the risk of vascular calcification in ESRD patients; see, e.g., Slatopolsky et al., N Engl J Med 315:157-161, 1986; Meric et al., Am J Kidney Dis 5:459-464, 1990. Hypercalcemia has been indicated in many serious side effects, such as cardiac arrhythmias, renal failure, and skin and visceral calcification. Research continues to address the concerns regarding the adverse effects associated with the calcium- or aluminum-based phosphate binders and to look for alternative agents for phosphorous control. And importantly, phosphate binder treatment is only marginally effective due, in part, to issues of patient compliance, as patients often end up having to take many tablets of said binders to adequately bind the ingested phosphate from foods.

Previous studies have evaluated nicotinic acid, and its metabolite, nicotinamide as a potential agent for phosphorous control. The biologic function of nicotinamide derives from its active form, nicotinamide adenine dinucleotide (NAD). NAD is proposed to be an intracellular regulator of sodium-dependent phosphate transport. Nicotinamide stimulates biosynthesis of NAD, inhibits catabolism of NAD, and increases the ratio of NAD (oxidized) to NADH (reduced); see, Kempson et al., J Clin Invest 67:1347-1360, 1981. Takahashi et al., Kidney Int 65:1099-1104, 2004, reported that nicotinamide significantly reduced serum phosphorous levels in hemodialysis patients, did not change serum calcium levels, and increased serum HDL cholesterol and decreased LDL cholesterol. Sampathkumar, et al., Int Urol Nephrol 38(1):l71-174, 2006, evaluated extended release nicotinic acid in hemodialysis patients with hyperphosphatemia and concluded that oral nicotinic acid may emerge as an agent for phosphorous control in dialysis patients. The mechanism by which nicotinamide reduces serum phosphorus levels remains unknown. Preliminary data from these studies indicate that the agent was well tolerated with few adverse effects, but further studies on adverse effects associated with long term administration are needed.

Related cardiac disease is one of the most important issues in ESRD patients on HD. The HD patient is often marked by constant malnutrition, inflammation, and atherosclerosis, resulting in a greatly increased risk of cardiovascular morbidity, and cardiovascular disease (CVD) remains a leading cause of death in patients with CKD stages 4 and 5. A large percentage of patients with CKD have traditional cardiac risk factors such as diabetes, hypertension and abnormalities in cholesterol. In the general population, interventions to address such traditional factors (e.g., intake of dietary antioxidants) have significantly decreased cardiovascular mortality. Unfortunately, however, the severity and extent of cardiovascular complications in patients with CKD is disproportionate to the number and severity of traditional risk factors and such interventions have not shown such benefit in the CKD population. This realization has thus focused more attention on non-traditional cardiac risk factors that are particularly relevant to patients with CKD, including for example, decreased hemoglobin levels, microalbuminuria, abnormalities in bone and mineral metabolism, and increased inflammation and oxidative stress.

As kidney disease progresses, oxidative stress appears to increase and correlates with the decline of renal function. Oxidative stress contributes to an environment where uremia may promote calcium phosphorus deposition in the arteries. Due to this ossification, endothelial dysfunction is thought to be one of the major factors responsible for the increased cardiovascular morbidity and mortality in CKD patients on dialysis.

Patients with impaired renal function have varying degrees of underlying inflammation. The cause of increased inflammation in ESRD patients is multifactorial and includes patient-related factors such as underlying disease, co-morbidity, oxidative stress, infections, malnutrition, genetic or immunologic factors; and on the other side, factors related to the HD procedure itself (e.g., membrane biocompatibility and dialysate quality). In the HD population, there is evidence that malnutrition of diets containing essential micronutrients is associated with chronic inflammation.

Many emerging and non-traditional predictive biomarkers have been recently studied in order to better understand the causes of inflammation and other non-traditional cardiac risk factors and the potential for their treatment and/or prevention. Examples of such biomarkers include: C-reactive protein (CRP), which is a surrogate marker for acute phase reactants and is used to monitor acute disease activity and tissue injury, has evaluated levels in the ESRD population due to the generation of pro-inflammatory cytokines, such as interleukin-1 (IL-1), interleukin-6 (IL-6), tumor necrosis factor alpha (TNF-a) and fibrinogen; advanced oxidation protein products (AOPP), a plasma protein biomarker used to assess oxidative stress, have elevated levels in patients with CKD on dialysis; advanced glycation end products (AGE) are known to be elevated in patients with CKD and these compounds possess pro-oxidant, pro-inflammatory and anti-endothelial properties; and asymmetric dimethylarginine (ADMA), an inhibitor of nitric oxide synthase (NOS), has been identified as a marker for cardiovascular risk in HD patients.

Gamma tocopherol, a Vitamin E analogue, has been found to be a potent antioxidant and its anti-inflammatory effect may be enhanced for patients with impaired renal function. In fact, gamma tocopherol already demonstrated to have efficacy in reducing inflammation in ESRD using a regimen of 300 mg po qd for 14 days; Himmelfarb et al., Kidney Int., 64: 978-991, 2003. At this dose in this population, in 15 patients treated for 14 days, no serious adverse events were reported; Id.

α-Lipoic acid (5-[(3R)-dithiolan-3-yl]pentanoic acid)(also referred to herein as ALA) is another compound that occurs naturally in some foods which may have some anti inflammatory properties. In fact, in some foods with ALA have been able to improve inflammatory profiles in patients; Maczurek A et al., Adv Drug Deliv Rev., 60(13-14):1463-70, 2008 Oct-Nov (Epub 2008 July 4); Bloedon et al., J Am Coll Nutr., 27(1):65-74, 2008. Described side effects include headache, rash, paresthesias, and muscle cramps. Isolated episodes of hypoglycemia have also been reported.

Supplementation with L-arginine and/or L-citrulline has been shown to restore vascular function by overcoming the deleterious effects of ADMA; Boger at al., Alternative Medicine Review 10(1):14-23, 2005, and references cited therein.

It has also been suggested that treatment with 5-methyltetrahydrofolate (5-MTHF) seems to induce an increase in survival rate by inducing a lower inflammatory state in ESRD patients see, e.g., Cianciolo et al., Am J Nephrol 28:941-948, 2008.

There are currently no approved therapies for inflammation associated with ESRD. As such, there clearly still exists the need to provide HD patients with additional therapies to be used with conventional therapies to lower the inflammatory burden and improve HD outcomes.

### SUMMARY OF THE INVENTION

The goal of the present invention is to overcome the deficiencies of currently-available therapy options for patients having impaired renal function; more specifically, to provide novel compositions and improved methods for treating bone and mineral disorders, cardiovascular risk, and inflammation in a ESRD population on HD, thus improving HD outcomes. The compositions and methods of the present invention will improve upon current therapies by providing nutritional supplements and methods which utilize fewer tablets/capsules and require less frequent administration than the current therapies, thereby improving patient compliance. Moreover, the novel compositions and methods may allow for a reduction/elimination in the need to administer other therapeutics currently being used in the current therapies, thus providing an overall more cost-effective option for these patients. As such, the present invention provides novel compositions and methods comprising various active ingredients which have been prepared as a nutritional supplement (e.g., multivitamin pill) having specific dosages and using a specified dosing regimen.

In one aspect of the present invention, the method comprises administration of a multivitamin comprising cholecalciferol, gamma tocopherol, and α-Lipoic acid to a patient. In a one preferred embodiment, the composition would be formulated as a multivitamin supplement to be given orally once a day (as two capsules), and containing a total dose of 300 mg of gamma tocopherol, 600 mg of α-Lipoic acid and 1500 IU of cholecalciferol.

In another aspect of the present invention, the method comprises combination therapy comprising co-administration of a multivitamin comprising cholecalciferol, gamma tocopherol, and α-Lipoic acid and one or more active ingredients such as nicotinamide, 5-MTHF, L-citrulline, L-arginine and other antioxidants (vitamins C and E). The one or more active ingredients may be formulated as one or more capsules. In one preferred embodiment, cholecalciferol, gamma tocopherol, and α-Lipoic acid would be formulated as one multivitamin pill, and the one or more active ingredients as a separate pill or pills.

In another aspect of the present invention, the method comprises administration of multivitamin comprising vitamin D and nicotinamide to a patient. In a one embodiment, the composition would be formulated as a multivitamin and contain 300 mg of nicotinamide and 500 IU of a cholecalciferol per pill, and assume a dosing regimen of up to 3 pills per day (1 pill qam, 1 pill midday, 1 pill qpm).

In another aspect of a present invention, a multivitamin pill for treating bone and mineral disorders and inflammation in patients with impaired renal function is provided; said multivitamin pill comprising a therapeutically effective amount of cholecalciferol, α-lipoic acid, and gamma tocopherol; and optionally one or more active ingredients.

In an alternative embodiment of the present invention, the cholecalciferol, gamma tocopherol, and α-Lipoic acid, and one or more active ingredients will be formulated to be administered intravenously to said patients.

In another aspect of the present invention, a “renal supplement pack” is provided for convenient use by a patient with impaired renal function. In a preferred embodiment, the pack is a blister pack or a plurality of blister packettes, a blister package, a lidded blister or a blister card or packet; wherein the composition(s) is arranged or clustered in the blister pack or a plurality of blister packettes: (a) in a chrono-dosing arrangement or pattern; or (b) individually.

### DETAILED DESCRIPTION OF THE INVENTION

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The present invention provides novel compositions comprising various active ingredients which have been prepared as a nutritional supplement, e.g., multivitamin pill, to be used in improved methods comprising novel dosing regimens.

Examples of active ingredients which may be useful in embodiments of the present invention include, but are not limited to: nicotinamide; vitamins A, B, C, D, B.sub.1, B.sub.2, B.sub.6, B.sub.2, E, and K; alpha, gamma, beta and delta (mixed) tocopherols; alpha lipoic acid; alpha linoleic acid; unsaturated fatty acids; acetaminophen; adenosine antagonists; biotin; calcium; calcium carbonate; cholestyramine resin; citalopram; citric acid; cod liver oil; cortisone acetate; potassium and sodium; ferrous fumarate, gluconate and sulfate; folic acid; thiamin; pantothenic acid; riboflavin; pyridoxine; ibuprofen; indomethacin; 5-methyltetrahydrofolate (5-MTHF); magnesium carbonate; L-citrulline; L-arginine; salicylate; trisilocate; naproxen and its sodium salt; niacin; niacinamide; nicotine; omega-3 polyunsaturated fatty acids; salicylic acid; sesame oil; shark liver oil; sodium bicarbonate; citrate and fluoride. Mixtures of these agents and their esters or pharmaceutically acceptable salts, solvates, hydrates, and/or polymorphs used for appropriate therapies are also contemplated.

The active ingredients of the present invention are preferably combined into a pharmaceutical composition which may be in the form of a solid powder, caplets, tablets, lozenges, pills, capsules, or a liquid, and which may be administered alone or in suitable combination with other components. In one embodiment of the present invention, the active ingredients are combined to prepare a pharmaceutical composition specifically formulated to be administered intravenously to said patients.

Because of their ease in administration, caplets, tablets, pills, and capsules represent the most advantageous oral dosage forms. The active ingredients can be blended to form a single composition or can form multiple compositions which may be co-administered. A preferred dosage of the compositions of the present invention may consist of one or more tablets for human oral consumption. If more than one tablet is used, each individual tablet may be identical to the other tablets, or each may contain only some of the ingredients of the composition, so that the combination of the different tablets comprises a composition of the present invention. Dosage forms of the invention such as tablets, pills, caplets, gel tabs, capsules, liquids, nutritional shakes, and sustained release formulations, and the like can be formulated and prepared according to manufacturing techniques well known in the pharmaceutical industry.

In one aspect of the present invention, the combination therapy comprises co-administration of at least two active ingredients which are formulated as separate compositions, e.g., a multivitamin pill and a separate pill, and manufactured in a separate package or container, e.g., a blister package or plurality of packettes, lidded blister or blister card. In another aspect, the combination therapy comprises co-administration of at least two active ingredients which are formulated in the same composition, e.g., a multivitamin pill, and manufactured in the same package or container, e.g., a blister package or plurality of packettes, lidded blister or blister card or any combination thereof. The invention contemplates that doses may be given at intervals of once a day, twice a day, three times a day, once every other day, three times a week, twice a week, weekly, or every 2 weeks.

Typically, the compositions will further include pharmaceutically acceptable ingredients and carriers such as starches, sugars, diluents, granulating agents, lubricants, binders, surfactants, sweetening and flavouring agents, disintegrating agents, coating materials, preservatives, dyes, thickeners, adjuvants, stabilizers, regulators, emulsifiers, flow agents, absorbents, antimicrobial agents, and the like or mixtures thereof depending on the form of the composition employed.

Diluents, also referred to as “fillers,” are typically necessary to increase the bulk of a solid dosage form so that a practical size is provided for compression of tablets or formation of beads and granules. Suitable diluents contemplated for use in the compositions described herein include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Lubricants are used to facilitate tablet manufacture. Lubricants contemplated for use in the compositions described herein include, but are not limited to, natural or synthetic oils, fats, polyethylene glycol, talc, waxes, or fatty acid salts such as magnesium stearate.

Binders are used to impart cohesive qualities to a solid dosage formulation, and thus ensure that a tablet or bead or granule remains intact after the formation of the dosage forms. Binders contemplated for use in the compositions described herein include, but are not limited to, gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,1-lactide/glycolide. Other examples for polymers are water-soluble polymers such as polyoxaalkylenes(polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol and esters or -ethers thereof, poly-vinylimidazole, poly-vinylpyrrolidon, and natural polymers like chitosan.

Surfactants contemplated for use in the compositions described herein include, but are not limited to, lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Sweetening agents and flavoring agents contemplated for use in the compositions described herein include, but are not limited to, sucrose, fructose, lactose or aspartame, peppermint, oil of wintergreen or fruit flavors such as cherry or orange flavor.

Disintegrating agents are used to facilitate dosage form disintegration or “breakup” after administration. Disintegrants contemplated for use in the compositions described herein include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers.

Preservatives contemplated for use in the compositions described herein include methyl or propylparabens, sorbic acid, chlorobutanol, phenol and thimerosal.

If desired, tablets or pills may be sugar coated or enteric coated by standard techniques. Coating materials contemplated for use in the compositions described herein include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT™ (Roth Pharma, Westerstadt, Germany), wax, gelatin, shellac, and polysaccharides.

Quick dissolve tablets may also be prepared, for example, by mixing the composition with agents such as sugars and cellulose derivatives, which promote dissolution or disintegration of the resultant tablet after oral administration, usually within 30 seconds.

Soft gel or soft gelatin capsules may also be prepared, for example, by dispersing the composition in an appropriate vehicle (vegetable oils are commonly used) to form a high viscosity mixture. This mixture is then encapsulated with a gelatin based film using technology and machinery known to those in the soft gel industry.

Chewable tablets may also be prepared, for example, by mixing the compositions with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are well versed in the processes and the machinery used as the chewable dosage form is a very common dosage form in the pharmaceutical industry.

Compressed tablets may also be prepared, for example, by mixing the composition with excipients intended to add binding qualities to disintegration qualities. The mixture is either directly compressed or granulated and then compressed using methods and machinery quite well known to those in the industry.

Various forms of release are contemplated for use herein, including, without limitation, immediate release, extended release, pulse release, variable release, controlled release, timed release, sustained release, delayed release, long acting, and combinations thereof. Such delayed release and extended release compositions can be prepared according to methods readily known in the art. For example, slow release formulations can be prepared by complexing the compositions of the present invention with a film of a polymer which is insoluble in the acid environment of the stomach, and soluble in the basic environment of lower GI tract to provide a substantial constant and effective level of the active ingredient(s) in the blood plasma. Examples of such rate controlling polymers include, but are not limited to, hydroxypropylmethylcellulose (HPMC), ethylcellulose, and methylmethacrylate. Gastrosoluble polymers, such as Eudragit™ E100 or an enteric polymer such as Eudragit™ L100-55D (supplied by Rohm America) may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

Controlled release formulations may also be obtained through encapsulation of dispersed micro-particles or emulsified micro-droplets via known dispersion or emulsion coating technologies.

The compositions of the present invention are used in the manufacture of a medicament or pharmaceutical composition to provide novel and improved methods of treatment of patients with impaired renal function. Complications of impaired renal function include anemia, changes in blood sugar metabolism, congestive heart failure, ESRD, decreased functioning of white cells, decreased immune response, dementia, electrolyte abnormalities (e.g., hyperphosphatemia), encephalopathy, fractures, hemorrhage, increased infections, joint disorders, liver inflammation, liver failure, loss of blood form the GI tract, nerve damage, pericarditis, peripheral neuropathy, platelet dysfunction, ulcers, seizures, uremia, and weakening of bones. The patient may be suffering from chronic renal insufficiency or end-stage renal disease. In another aspect, the patient may be pre-dialysis. In a further aspect, the patient may be suffering from uremia. In another aspect, the patient can be suffering from diabetes mellitus I or II. In another aspect, the patient can be suffering from a cardiovascular disorder.

The term “hyperphosphatemia” as used herein means an electrolyte disturbance in which there is an abnormally elevated level of phosphate in the blood such that the phosphate concentration of serum is beyond a clinically defined normal region. Hyperphosphatemia is typically defined as possessing a serum phosphate levels of over about 6 mg/dL. The condition, especially if present over extended periods of time, leads to severe abnormalities in calcium and phosphorus metabolism and can be manifested by aberrant calcification in joints, lungs, and eyes. The compositions of the present invention can lower the phosphate concentration of serum and can inhibit phosphate transport and thus can be used for the prevention or treatment of hyperphosphatemia. In addition, because hyperphosphatemia secondarily leads to hypocalcemia, which may induce secondary hyperparathyroidism, it is understood that the compositions according to the present invention can be used for the prevention and treatment of secondary hyperparathyroidism as well.

The compositions of the present invention can also be used for the prevention and treatment of diseases which are induced by PTH increase in the secondary hyperparathyroidism, e.g., central or peripheral nervous system damage, anemia, myocardiopathy, hyperlipidemia, anomaly of saccharometabolism, pruritus cutaneus, tendon rupture, sexual dysfunction, muscle damage, skin ischemic ulcer, growth retardation, heart conduction disturbance, pulmonary diffusing impairment, immune deficiency, ostealgia and arthralgia, bone deformity, or fracture.

The compositions of the present invention can also be for the treatment of diseases such as age-related arterial sclerosis, diabetic vasculopathy, calcification of soft tissue, metastatic calcification, and ectopic calcification, and calcification of cardiovascular system in dialysis patients.

Dosages of the active ingredients in the compositions of the present invention will be dependent on the particular disease state being addressed. For example, higher doses of the active ingredients of the present invention are used where therapeutic treatment of a disease state (e.g., chronic kidney disease) is the desired end, while the lower doses are generally used for prophylactic purposes, it being understood that the specific dosage administered in any given case will be adjusted in accordance with the specific active ingredients being administered, the disease to be treated, the condition of the subject and the other relevant medical facts that may modify the activity of the drug or the response of the subject, as is well known by those skilled in the art. The compositions of the present invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period.

Nicotinamide contemplated for use as an active ingredient in the compositions of the present invention are widely available and understood by those skilled in the art. Dosages of nicotinamide contemplated for use include, but are not limited to, 300-400 mg of nicotinamide per pill. This would be in line with mean dose requirements for nicotinamide in clinical trials.

Vitamin D contemplated for use as an active ingredient in the compositions of the present invention includes cholecalciferol and ergocalciferol. Cholecalciferol and ergocalciferol are fat-soluble seco-steroid precursors to Vitamin D hormones that, among other activities, contribute to the maintenance of normal levels of calcium and phosphorus in the bloodstream; U.S. Patent Application No. 20070122477 (Bishop et al.). Cholecalciferol and ergocalciferol are normally present at stable, low concentrations in human blood. Both cholecalciferol and ergocalciferol are metabolized into prohormones by enzymes primarily located in the liver of the human body. Cholecalciferol is metabolized into a prohormone 25-hydroxyvitamin D.sub.3, and ergocalciferol is metabolized into two prohormones, 25-hydroxyvitamin D.sub.2 and 24(S)-hydroxyvitamin D.sub.2. The two 25-hydroxylated prohormones are collectively referred to as “25-hydroxyvitamin D” (“25(OH)D”). Cholecalciferol and ergocalciferol also can be metabolized into prohormones outside of the liver in certain cells, such as enterocytes, by enzymes which are identical or similar to those found in the liver.

Dosages of cholecalciferol and ergocalciferol (separately or combined) contemplated for use in oral dosage forms include at least 500 IU, 1000 IU, 1,500 IU, or at least 2,000, 2,500, 3,000, 4,000, 5,000, 6,000, 7,000, 7,500, 8,000, 9,000, 10,000, 11,000, 12,000 or 12,500 IU. The invention contemplates that doses may be given at intervals of once a day, twice a day, three times a day, once every other day, three times a week, twice a week, weekly, or every 2 weeks. The recommended safe range for ergocalciferol has been described as 1,000 to 2,000 IU/day in renal failure patients, although doses as high as 50,000 IU monthly have been used. The cumulative dose taken each time may be 1,500 IU (cholecalciferol and ergocalciferol separately or combined), or at least 2,000, 2,500, 3,000, 4,000, 5,000, 6,000, 7,000, 7,500, 8,000, 9,000, 10,000, 11,000, 12,000 or 12,500 IU. Such doses are preferred for use with adult humans. In one embodiment of the present invention, the dose will be a total of 1500 IU of cholecalciferol provided in 2 capsules to be taken once per day.

Antioxidants contemplated for use in preferred embodiments include tocopherols and alpha lipoic acid and/or alpha linoleic acid. Dosages of tocopherols contemplated for use include, but are not limited to, 200-400 mg of tocopherols provided in 2 capsules to be taken once per day. Dosages of alpha lipoic acid and/or alpha linoleic acid contemplated for use include, but are not limited to, 100-300 mg per pill. In one embodiment of the present invention, the dose will be a total of 600 mg alpha lipoic acid provided in 2 capsules to be taken once per day.

Dosages of L-citrulline and L-arginine contemplated for use include, but are not limited to, oral 1-10 gm/day. Other doses contemplated for use are described in the references cited herein.

Dosages of 5-MTHF contemplated for use include, but are not limited to, 50 mg iv, administered at the end of a HD session, or oral 5-25 mg/day administration. Other doses contemplated for use are described in the references cited herein.

The resultant compositions of the present invention may conveniently be presented in unit dosage form and may be prepared by conventional pharmaceutical techniques, according to market need, i.e., unit dose, rolls, bulk bottles, blister-packs, etc. Blister-pack medication packages are readily available and well known in the art. The present invention contemplates the use of a “renal supplement package” comprising a plurality of chambers for containing pills or tablets to be used by the patient in his/her therapy regimen.

### EXAMPLE 1

In this example, various combinations of active ingredients are evaluated in a prospective study to evaluate the effectiveness of a novel renal multivitamin on Vitamin D levels, EPO dose, inflammatory (e.g., C-reactive protein) and other biomarkers in an ESRD population on HD. Study drug was manufactured by a contract manufacturer using GMP processes and delivered to the site.

Specifically, three active ingredients (cholecalciferol, α-lipoic acid, and gamma tocopherol) are evaluated in a 12 week study comprising the following method: the active ingredients are formulated as two capsules to be administered orally once daily to provide a total daily dose of cholecalciferol at 1500 IU, gamma tocopherol at 300 mg, and α-Lipoic Acid at 600 mg. This is a fixed dose study with no titration.

The study is a total of 13 weeks including a 1 week screening period. Efficacy of the multivitamin on Calcidiol levels (Vitamin D levels) is the primary endpoint. Comparisons will be made between start of week 1 and end of week 12 readings. Change in EPO dose, hemoglobin and CRP levels are the secondary endpoints and comparisons will be made between start of week 1 and end of week 12 readings.

### EXAMPLE 2

In this example, four active ingredients (nicotinamide, cholecalciferol, alpha lipoic acid, and gamma tocopherol) are evaluated in a study comprising the following combination therapy method: 1) administration of a multivitamin pill (comprising 300 mg of nicotinamide, 500 IU of a cholecalciferol, and 200 mg alpha lipoic acid per pill) to be administered three times a day (1 pill qam, 1 pill midday, 1 pill qpm); and 2) a stand alone pill comprising 300 mg gamma tocopherol to be administered qam with said multivitamin pill. Overall efficacy of this combination therapy on markers of bone and mineral metabolism (e.g., serum phosphorous levels) and on markers of inflammation, C reactive protein (CRP) and IL-6 in an ESRD patient population is evaluated based upon comparisons to be made between the start of and end of study readings.

What is claimed is:

1. A method for treating bone and mineral disorders and inflammation in patients with impaired renal function, said method comprising orally administering to said patient a pharmaceutical composition comprising a therapeutically effective amount of vitamin D, α-lipoic acid, and gamma tocopherol; and optionally one or more active ingredients.

2. The method of claim 1, wherein said vitamin D is cholecalciferol.

3. The method of claim 2, wherein said pharmaceutical composition is formulated as a multivitamin pill.

4. The method of claim 1 wherein said one or more active ingredients is selected from the group consisting of 5-MTHF, L-arginine, L-citrulline, nicotinamide, and alpha linoleic acid.

5. A method for treating bone and mineral disorders and inflammation in patients with impaired renal function, said method comprising orally administering to said patient a pharmaceutical composition comprising a therapeutically effective amount of vitamin D and nicotinamide; and optionally one or more active ingredients.

6. The method of claim 5, wherein said pharmaceutical composition is formulated as a multivitamin pill.

7. A multivitamin pill for treating bone and mineral disorders and inflammation in patients with impaired renal function, said multivitamin pill comprising a therapeutically effective amount of cholecalciferol, α-lipoic acid, and gamma tocopherol; and optionally one or more active ingredients.

8. A renal supplement pack comprising a blister pack or a plurality of blister packettes, a blister package, a lidded blister or a blister card or packet; wherein the composition(s) is arranged or clustered in the blister pack or a plurality of blister packettes: (a) in a chrono-dosing arrangement or pattern; or (b) individually.
